(19) United States
(12) Patent Application Publication
(10) Pub. No.: US 2026/0060818 A1
(43) Pub. Date: Mar. 5, 2026

(54) CUFF AND ORTHOTIC SYSTEM

(71) Applicant: macu4 AG, Zurich (CH)

(72) Inventors: Lukas Martin SCHILLER, Zurich (CH); Alec Corentin CHEVROT, Chavannes-près-Renens (CH)

(21) Appl. No.: 18/821,085

(22) Filed: Aug. 30, 2024

(51) Int. Cl. A61F 2/58; A61F 2/54; A61F 2/78; A61F 5/01

(52) U.S. Cl. CPC A61F 2/588; A61F 2/78; A61F 5/0118; A61F 2002/543

(57) ABSTRACT

A cuff combinable with accessories for forming an orthotic system comprises a cuff base in form of a lower shell and a fixation element combinable into one sleeve attachable to a limb through direct or indirect interlocking. The lower shell and the fixation element provide a system that can be tightened around the limb by reducing a circumference formed by the lower shell and the fixation element, such that load transfer from an accessory to the limb is enabled. First and second magnetic parts are provided in a body of the lower shell and in or at the fixation element. By means of an attracting magnetic force between both magnetic parts the fixation element can be moved onto one side of the body of the lower shell or can be held at one side of the body of the lower shell. An orthotic system is also disclosed.

The present disclosure relates to a cuff as well as to an orthotic system. The orthotic system comprises accessories to serve besides others general assistance of the griping or replacing the griping. The orthotic system is also called an ortho-prosthetic system.

### BACKGROUND

In the case of a congenital deformity or amputation, it may be difficult to perform bimanual activities such as cycling. Similarly, holding a shopping bag can become a challenge. Daily life for these people is full of little challenges that an ordinary person would never suspect, let alone imagine. People with a birth defect learn to live with it, but outside help is welcome to ease the effort and mental burden. For people who have suffered an amputation as a result of an accident, for example, it's necessary to put a relearning of skills in place. Tools can facilitate not only their daily life but also their rehabilitation. Similarly, in the event of an accident, surgery or injury, hands may become weakened, sensitive, immobilized for a limited period. Therefore, a rapid provision of support is essential to enable affected individuals to continue their activities and daily routines.

### SUMMARY

An orthotic system is designed for individuals with partial or complete loss of hand function. This loss may be due to temporary or permanent weakening, hand injury, paralysis, neuromuscular disease, partial absence of the hand or absence due to wrist disarticulation.

An orthotic system designed for forearms is intended to assist individuals with a disability below the wrist. This encompasses various scenarios, including those with only a portion of the palm remaining, small residual fingers, a limited number of fingers, or limited and painful movement capacity. In consequence, the system should be engineered to transmit the force generated by the accessory directly to the forearm.

Existing solutions are often poorly adapted, expensive and restricting in terms of the activities they support.

The problem to be solved by the present invention is to provide a cuff and an orthotic system that ensure comfort through a lightweight, intuitive and modular design, while transferring load forces that occur in the area of the accessories directly onto a limb.

This problem is solved by the subject-matter of the independent claims.

Embodiments of the invention are claimed in dependent claims.

A first aspect of the invention relates to a cuff, which is combinable with accessories to form an orthotic system, comprising a cuff base in form of a lower shell and a fixation element, which can be combined or is combined with the lower shell into a sleeve attachable to a limb through direct or indirect interlocking with each other, wherein the lower shell and the fixation element provide a system that can be tightened around the limb by reducing a circumference formed by the lower shell and the fixation element, such that load transfer from an accessory to the limb is enabled.

The cuff can be designed to be positioned on an arm, for instance on a forearm or on an upper arm.

In an embodiment of the cuff the interlocking between the lower shell and the fixation element comprises a magnetic connection, wherein in a body of the lower shell at least one first magnetic part is provided and in or at the fixation element at least one second magnetic part is provided such that by means of an attracting magnetic force between both magnetic parts the fixation element can be moved onto one side of the body of the lower shell or is or can be held on one side of the body of the lower shell.

The first magnetic part and the second magnetic part do not necessarily both have to be made of a magnetic material, it is sufficient that at least one of the two magnetic parts is made of or comprises a magnetic material, and the other magnetic part can be either made of, or comprise, a magnetisable or magnetic material.

The lower shell may comprise at least one first form fit means and the fixation element may comprise at least one second form fit means, wherein both form fit means are adapted to realize a form fit connection.

The form fit connection may provide an undercut in order to prevent its release of the form fit connection when subjected to a force that is essentially perpendicular to the insertion direction of the form fit connection.

The magnetic parts pull the lower shell and/or the fixation element into positions in which the form fit connection is established.

Alternatively, the magnetic parts pull the lower shell and/or the fixation element into positions still spaced apart from each other, so that no form fit connection is established merely by means of the magnetic force.

This facilitates an easy decoupling of the form fit connection. When there is tension in form fit means, the form fit means are pulled into the positions where the form fit connection happens.

In one embodiment of the cuff, the fixation element is an upper shell.

Each shell is moulded as an ergonomically adapted cavity, which aligns with the natural shape of the human limb.

The advantage of the fixation element in form of an upper shell is that it significantly increases stability.

Particularly, in this embodiment the cuff comprises at least two shells that can be combined to form one all-around coverage of the limb.

The invention is not limited to the upper and lower positions of the upper shell and the lower shell, but these shells can also be located at other positions on the limb.

The cuff may provide at least one interlocking member in form of at least one lacing element, which is mechanically connected to both shells when the interlocking member is interlocked, wherein the lacing element is adapted in such a way that when shortening an effective length of the lacing element, the distance between the two shells is reduced.

The lacing element may comprise one of the second magnetic parts and/or one of the second form fit means, in order to provide the magnetic connection and/or the form fit connection. By manipulating the lacing system, the lacing element is shortened, thereby generating a pulling force within the lacing element, which is transmitted to the second form fit means, which is in turn transferred from the second form fit means to the first form fit means and subsequently into the cuff base or lower shell in order to pull the cuff base or lower shell towards the cuff top or upper shell.

A ratchet fixation system could also be considered as alternative to the lacing system.

In another embodiment of the cuff, the fixation element is a strap system.

The strap system corresponds to the interlocking member. A strap of the strap system can be directly mounted through a slit on the cuff base.

The advantage of a strap system is its high breathability.

It is not excluded from the scope of the present invention that the strap system and the upper shell are used simultaneously.

The strap system may comprise at least one of the second magnetic parts and/or one of the second form fit means.

In an embodiment, where the fixation element is an upper shell, and/or in an embodiment in which the fixation element is a strap system, the first form fit means can be at least one geometric interlocking piece, for instance in the form of a hook, which serves as a connection between the two shells or serves as a connection between the lower shell and the strap system, wherein the geometric interlocking piece may be incorporated into the lower shell.

Aforementioned first form fit means or geometric interlocking piece may exist as a separate body, either distinct from both shells or solely from the lower shell and the strap system, respectively.

Aforementioned first form fit means or geometric interlocking piece may be connectable geometrically on one side with the lower shell and linked to the upper shell by the lacing element and its second form fit means.

Aforementioned form fit means or geometric interlocking piece may be attracted to its fitting and held in place by magnetic force generated by the magnetic parts.

At least one strap of the strap system may comprise a hook-and-loop fastener.

The use of such a hook-and-loop fastener simplifies the attachment and the removal of the cuff for the user.

The cuff can be intended for persons with partial or complete loss of the hand function due to temporary or permanent weakening, hand injury, paralysis, neuromuscular disease, partial absence of the hand or absence due to wrist disarticulation.

Through the utilization of the cuff with an appropriate accessory, a lack of gripping dexterity, capability or force can be reacquired or supported. It aims to support the activities of the user such as sports, leisure and daily routines.

The cuff corresponds to the part which comes in direct contact with the limb, in particular the forearm, of the user.

Thereby, the cuff can be operated with one hand, its design is user friendly and ergonomic.

Alternatively, at least one strap of the strap system may comprise at least an elastic strip that can be tensioned and adapted in shape to the shape and size of the limb.

A further aspect of the present invention is an orthotic system, comprising a described cuff and a module mechanically connected or connectable to the cuff, wherein the cuff comprises a first part of a ratchet system and the module comprises a second part of the ratchet system, wherein the first part and the second part have form elements, respectively, which are complementary in form and size.

One of the elements of the cuff and module may comprise a retainer, equipped with one of the first part or second part of the ratchet system and the respective other element of the cuff and module may comprise an insertion element that is intended to be positioned in the retainer, wherein the insertion element is equipped with the respective other part of the first part or second part of the ratchet system and is designed to be pushed into the retainer along a slide-in direction, and due to an application of force to bring the first parts and second parts in a positive locking.

The application of force may be realized by an elastic behaviour of the insertion element that is pushing the respective first part or second part of the ratchet system in the direction of the respective first part or second part at the retainer in order to establish a positive locking between the first parts and the second parts.

The insertion element may be bent while inserting the insertion element into the retainer. Thereby the retainer is configured to absorb the induced bending moment, enabling the application of force needed to establish a positive locking.

The elasticity of the insertion element can be adapted in such a way that the insertion element can be manually pushed against its elastic force and thus be moved by applying a pressing force of at least 3 N, thereby disengaging the first part and second part of the ratchet system from the positive locking. This allows the user to manually remove the positive locking if desired.

The push direction is essentially perpendicular to the slide-in direction.

A plurality of first parts and second parts may be arranged in rows, respectively, so that the positive locking can be established at different positions along the slide-in direction.

At least one of the elements of the orthotic system may be manufactured by additive manufacturing and, more specifically, selective laser sintering. The material can be either PA11 or PA12. Depending on the volume of this product line, another manufacturing technique, such as injection moulding, could be considered, too, as well as other materials.

The prosthetic system may be combined with various accessories, enabling the user to pursue activities such as riding a bike, holding a bag, pulling a suitcase, walking with crutches, kayak or ski.

### BRIEF DESCRIPTION OF THE DRAWINGS

The invention is further illustrated by the following examples and figures, from which further embodiments and advantages can be drawn. These examples are meant to illustrate the invention but not to limit its scope.

It is shown in

FIG. 1: a first embodiment of the cuff in a perspective view,

FIG. 2: a partial sectional view of the cuff shown in FIG. 1 before form fit connection,

FIG. 3: the partial sectional view of the cuff shown in FIG. 1 in form fit connection,

FIG. 4: a further embodiment of the cuff shown in FIG. 2 in form fit connection,

FIG. 5: a second embodiment of the cuff in a perspective view,

FIG. 6: the underside of the cuff shown in FIG. 1,

FIG. 7: an orthotic system in a perspective view,

FIG. 8: a part of the lower shell as well as a part of the insertion element in a first state,

FIG. 9: the part of the lower shell as well as the part of the insertion element in a second state,

FIG. 10: the part of the lower shell as well as the part of the insertion element in a third state, and

FIG. 11: the part of the lower shell as well as the part of the insertion element in a fourth state.

### DETAILED DESCRIPTION

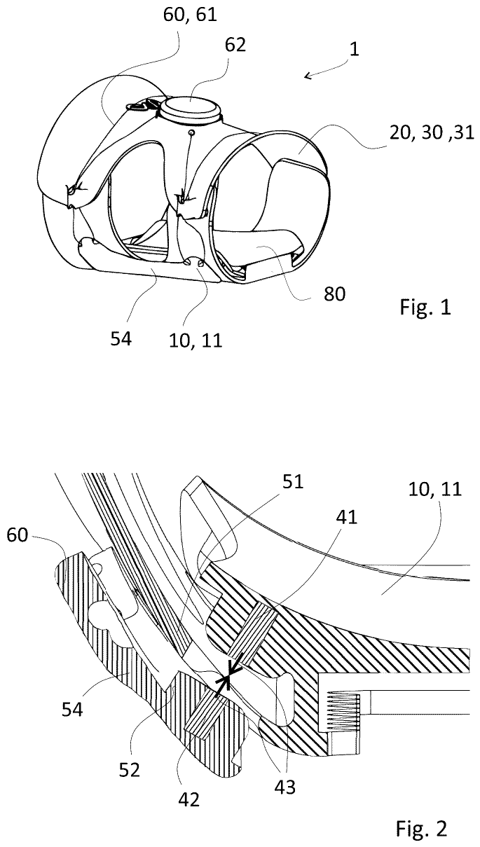

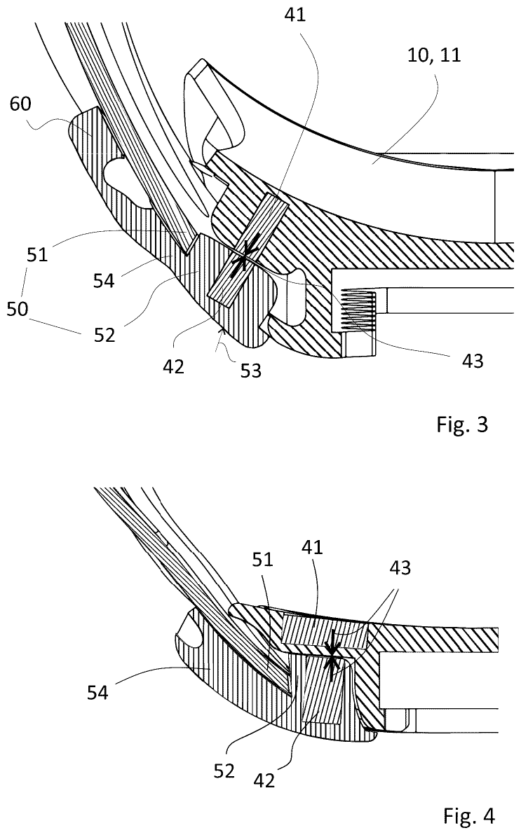

Insertion of a limb, for instance a forearm, into the cuff 1 can be difficult as, in most cases, there is an enlargement at the beginning of the palm. This makes it difficult to insert the arm into the cuff 1 from the proximal side. In order to facilitate the insertion of the arm into the cuff 1 on the proximal side, the design is divided into a cuff base 10 in form of a lower shell 11 and a fixation element 20, which can be a cuff top 30 in form of an upper shell 31, see FIG. 1, or a strap system 70, see FIG. 3.

As shown in the embodiment of FIG. 1, the shells 11, 31 can be separated as two distinct elements. The lower shell 11 can in this case be set radially to the axis of the arm. The upper shell 31 can then be coupled to the lower shell 11 to create a closed system around the forearm.

The number of shells 11, 31 can be larger than two and rise to three, four or more, but that can be the case only if they can be linked in such a way that the user does not need to hold them all in position before tightening them together around their forearm.

The way in which the shells can be connected to each other should be easy and effortless. In the embodiment shown in FIG. 1, the upper shell 31 as the fixation element 20 is connected to the lower shell 11 by means of the lacing system 60, which comprises second form fit means 52, which is adapted to allow a geometrical interlocking with first form fit means 51 of the lower shell 11 on demand. Through this, the second form fit means 52 and the first form fit means create a form fit connection 50.

This means that the second form fit means 52 can be disconnected from the lower shell 11 whenever it is desired. The second form fit means 52 acts as a hook.

Each second form fit means 52 is provided at a lacing element 61 in form of a cable, which is connected with a lacing knob 62. By turning of the lacing knob 62, tension is generated in the lacing element, which causes the tightening of the lacing system 60.

As the tension generated in the lacing system 60 increases, the circumference becomes reduced, allowing the lacing system 60 to be tightened around the forearm which results in better transmission of the forces exerted on the cuff 1.

The fixation element 20 in form of the upper shell 31 comprising the lacing system 60 may comprise according to the embodiment shown in FIG. 1 a common part 54 at which a plurality of the second form fit means 52 are provided. Thus, at the lower shell 11 a number of the first form fit means 51 is provided accordingly.

As shown in FIGS. 2 to 4, in order to increase the user friendliness, at least one first magnetic part 41 is positioned in the body of the lower shell 11, and at least one second magnetic part 42 is positioned in the second form fit means 52 at the lacing system. In the case of the use of a common part at which a plurality of the second form fit means 52 are provided, a corresponding number of second magnetic parts 42 is provided at this common part 54, too.

Thus, the second magnetic part 43 and therefore the hook of the second form fit means is guided by the attracting magnetic force 43 of both magnetic parts 41, 42 into the first form fit means 51 in the lower shell 11 along the insertion direction 53, shown in FIG. 2.

The magnetic parts 41, 42 then hold the form fit means 51, 52 in place by means of the attracting magnetic force 43, therefore creating a magnetic connection 40, see FIG. 3. Once tension is set in the lacing system 61, the magnetic parts 41, 42 no longer play a role, since the form fit means 51, 52 are geometrically interlocked, for instance by means of an undercut, shown in FIG. 2.

Once the form fit means 51, 52 are in place in the lower shell 11, the lacing system 60 can start to be operated. The higher the cable tension, the more difficult it becomes to remove the form fit means 51, 52. The cuff top 30 slides on the cuff base 10 when the lacing system 60 gets tightened.

The number of form fit means 51, 52 can vary, and the form fit means 51, 52 can be positioned at different positions, as shown in FIG. 4. The interlocking form fit means 51, 52 can also be integrated into the shells, in this case the shells must be deformed when the lacing system 60 is being operated.

In order to remove the cuff 1, the user must release the tension of the lacing system 60 and pull radially on the second form fit means 52, or on the lacing element 61. The cuff 1 can be removed, once the second form fit means 52 is released.

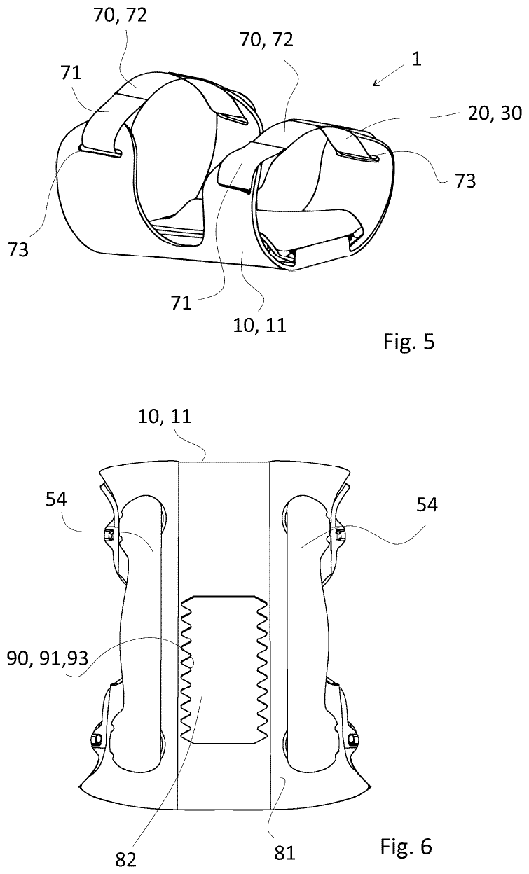

In FIG. 5, a different embodiment of the cuff 1 is shown. In this embodiment, the cuff 1 provides a cuff base 10 in form of a lower shell 11 and a fixation means 20 in form of a strap system 70. The strap system 70 comprises two straps 71 which are led through slits 73 of the cuff base 10. The straps 71 are provided with a hook-and-loop fastener 72, respectively.

By tensioning the straps 71 and fixation of the hook-and-loop fasteners 72, the strap system 70 can be tightened around the forearm.

The embodiment of the cuff 1 comprising a strap system 70 is not restricted to the embodiment shown in FIG. 5.

A further embodiment not shown here may comprise a hook-based closure, similar or equal to the common part 54 at which a plurality of the second form fit means 52 and/or second magnetic parts 42 are provided and which is shown in FIG. 1.

When the user places the cuff base 10 on his arm, the second form fit means 52 approaches its position, thanks to the attracting magnetic force 43 of the magnetic parts 41, 42. The user can tighten the strap system 70 with one hand thereby achieving an adjusted tighter or looser fit of the cuff 1 on the arm.

In both embodiments shown in FIGS. 1 and 3, cushioning material 80 is applied in the cuff base 10.

Another aspect of the design is its modularity.

The system allows for the connection of different modules to best meet the needs of the users. The module 100 can be inserted into the lower shell 11 of the cuff 1.

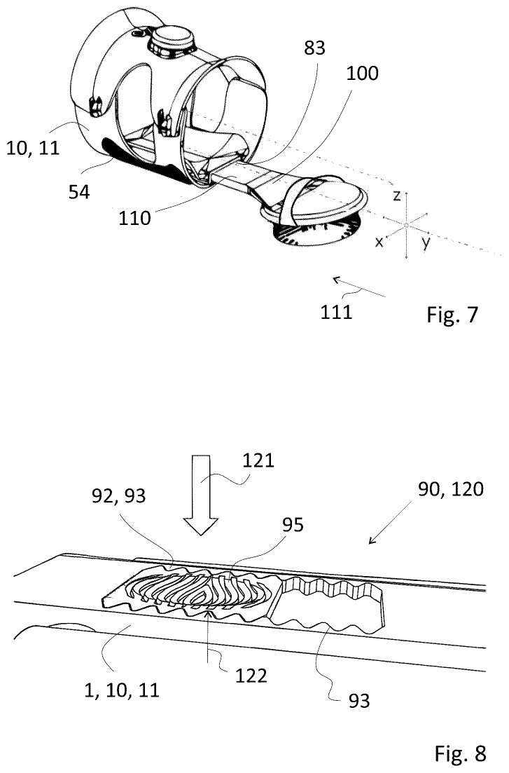

As shown in FIG. 7, an insertion element 110 of a module 100 can be directly inserted along a slide-in direction 111 into a retainer 82, which is provided in the cuff base 10 through an insertion opening 83 which in turn is arranged at the distal end of the cuff 1, shown in FIG. 6.

Once the insertion element 110 of the module 100 is inserted in the insertion opening 83, the user has to press a button 95, shown in FIGS. 8 to 11, and slide the insertion element 110 of the module 100 into the lower shell 11, while keeping the button 95 pressed. Here, the button 95 is a special design on the insertion element 110 of the module 100, which slides into the lower shell 11. The user can then release the button 95 when the desired position is reached.

For fixation of different positions of the module 100 with regard to the cuff 1, the orthotic system comprising the cuff 1 and the module 100 further comprises a ratchet system 90. The ratchet system 90 comprises a first part 91 provided at the lower shell 11 of the cuff 1, as well as a second part 92 provided at the insertion element 110 of the module 100 and/or at the button 95. Both components 91, 92 of the ratchet system 90 feature form elements 93, each having a shape like a tooth. In an embodiment, such tooth may have a rounded shape. The form elements 93 of the first part 91 and the second part 92 are complementary to each other in form and size.

Positive locking 120 of these form elements 93 allows the transfer of loads along the y-axis, shown in FIG. 7.

Furthermore, the module 100 can slide along the y-axis in the slide-in direction 111 parallel to an arm axis. Depending on the activity and intensity, different positions of the module 100 along the y-axis might be required. This allows for the same design to be used by users with different arm lengths.

The distance between the area of interest of the module 100 and the insertion opening of the lower shell 11 as seen in FIG. 7 can be adjusted by means of the ratchet system 90.

The number of positions of the module 100 is determined by the number of form elements 93 on the first part 91 and the second part 92 of the ratchet system 90.

FIGS. 8 to 11 show a method for changing the position of the module with respect to the cuff 1.

In FIGS. 8 to 11 a part of the lower shell 11 or the cuff base 10 of the cuff 1 is shown, as well as a part of the insertion element 110 of a module, which is not shown completely.

At the insertion element 110 the button 95 is provided.

In the retainer 82 of the lower shell 11 a first part 91 of the ratchet system 90 is provided, comprising a plurality of form elements 93 in the shape of tooth, respectively.

At the insertion element 110 and/or at the button a second part 92 of the ratchet system 90 is provided, comprising also a plurality of form elements 93 in the shape of a tooth, respectively.

The form elements 93 of the first part 91 and of the second part 92 of the ratchet system 90 are arranged in rows, respectively, and are complementary to each other in form and size.

FIG. 8 shows a state in which the form elements 93 of the first part 91 and the second part 92 engage with each other. Thus, a positive locking 120 is established between the lower shell 11 and the insertion element 110. This engaged state is maintained by means of a force 122 acting on the insertion element 110 due to elasticity of the insertion element 110.

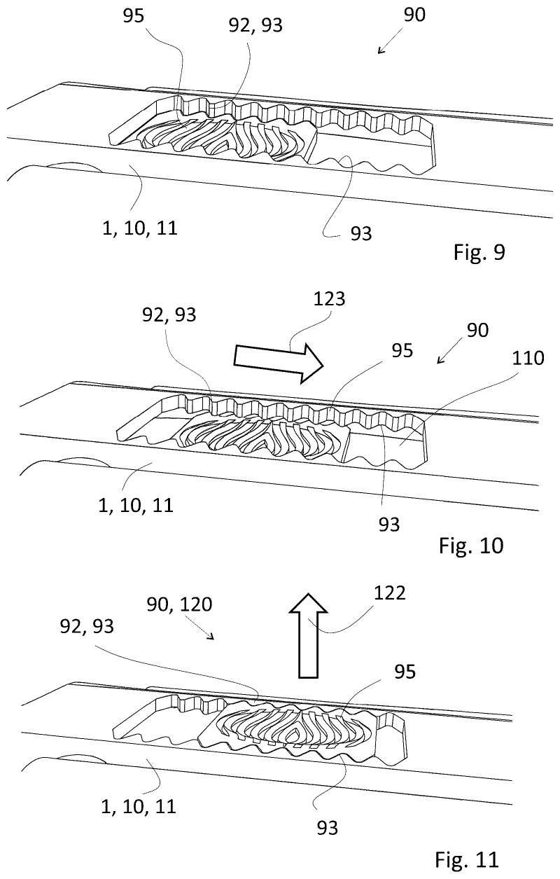

When the button 95 is pressed by a pressing force 121, for instance manually, the button 95 and therefore the insertion element 110 is pressed inward against the elastic force 122, wherein the form elements 93 of the first part 91 and of the second part 92 of the ratchet system 90 are moved out of their engagement, so that the positive locking 120 does not exist anymore, as shown in FIG. 9.

When the insertion element 110 has been moved inward, by applying a moving force 123 on the button 95, the insertion element 110 can be moved parallel to the extension of the rows of form elements 93 of the first part 91 and of the second part 92 of the ratchet system 90. Thus, the position of the insertion element 110 and therefore the position of the module with respect to the cuff 1 can be adjusted, see FIG. 10.

When the foreseen position has been reached, the button 95 can be released and due to elastic force 122 the insertion element 110 as well as the button moves so that the form elements 93 of the first part 91 and of the second part 92 of the ratchet system 90 engage again, shown in FIG. 11.

The module can be removed from the cuff, when necessary, for example, if the targeted activity changes, requiring another module.

The system allows for outdoor activities such as cycling and can also be used in the water, for example, when kayaking. Both of these activities require a critical degree of freedom so as not to restrict the movement of the upper limbs. The system then allows for symmetrical body development through the activities being practised. Furthermore, it can be used for muscle-strengthening sessions or fitness training.

### LIST OF REFERENCE SIGNS

1 Cuff
10 cuff base
11 lower shell
20 fixation element
30 cuff top
31 upper shell
40 magnetic connection
41 first magnetic part
42 second magnetic part
43 attracting magnetic force
50 form fit connection
51 first form fit means
52 second form fit means
53 insertion direction
54 common part
60 lacing system
61 lacing element
62 lacing knob
70 strap system
71 strap
72 hook-and-loop fastener
73 slit
80 cushioning material
81 distal end
82 retainer
83 insertion opening
90 ratchet system
91 first part of the ratchet system
92 second part of the ratchet system
93 form element
95 button
100 module
110 insertion element
111 slide-in direction
120 positive locking
121 pressing force
122 force
123 moving force

1. A cuff combinable with accessories for forming an orthotic system, comprising a cuff base in form of a lower shell and a fixation element which can be combined or is combined with the lower shell into a sleeve attachable to a limb through direct or indirect interlocking with each other, wherein the lower shell and the fixation element provide a system that can be tightened around the limb by reducing a circumference formed by the lower shell and the fixation element, such that load transfer from an accessory to the limb is enabled, wherein the interlocking between the lower shell and the fixation element comprises a magnetic connection, wherein in a body of the lower shell at least one first magnetic part is provided and in or at the fixation element at least one second magnetic part is provided such that by means of an attracting magnetic force between both magnetic parts the fixation element can be moved onto one side of the body of the lower shell or is or can be held at one side of the body of the lower shell.

2. The cuff according to claim 1, wherein the lower shell comprises at least one first form fit means and the fixation element comprises at least one second form fit means, wherein both form fit means are adapted to realize a form fit connection.

3. The cuff according to claim 2, wherein the form fit connection provides an undercut in order to prevent release of the form fit connection when subjected to a force essentially perpendicular to an insertion direction of the form fit connection.

4. The cuff according to claim 1, wherein the fixation element is an upper shell.

5. The cuff according to claim 4, wherein the cuff provides at least one interlocking member in form of at least one lacing element, which is mechanically connected to both shells when the interlocking member is interlocked, wherein the lacing element is adapted in such a way that when shortening an effective length of the lacing element, a distance between the two shells is reduced.

6. The cuff according to claim 5, wherein the lacing element comprises one of the second magnetic parts and/ or one of the second form fit means.

7. The cuff according to claim 1, wherein the fixation element is a strap system.

8. The cuff according to claim 7, wherein the strap system comprises at least one of the second magnetic parts and/ or one of the second form fit means.

9. The cuff according to claim 7, wherein at least one strap of the strap system comprises a hook-and-loop fastener.

10. The cuff according to claim 8, wherein at least one strap of the strap system comprises a hook-and-loop fastener.

11. The orthotic system, comprising a cuff according to claim 1 and a module mechanically connected or connectable to the cuff, wherein the cuff comprises a first part of a ratchet system and the module comprises a second part of the ratchet system, wherein the first part and the second part have form elements, respectively, which are complementary in form and size.

12. The orthotic system according to claim 11, wherein one of the elements cuff and module comprises a retainer provided with one of the first part or second part of the ratchet system and the respective other element of cuff and module comprises an insertion element for positioning in the retainer, wherein the insertion element is provided with the respective other part of the first part or second part of the ratchet system and is designed to be pushed into the retainer along a slide-in direction, and due to an application of force to bring the first part and second part in a positive locking.

13. The orthotic system according to claim 12, wherein the application of force is realized by an elastic behaviour of the insertion element pushing the respective first part or second part of the ratchet system in the direction of the respective first part or second part at the retainer in order to establish the positive locking, wherein the Young's modulus of the insertion element is between 0.5 GPa and 7 GPa.

14. The orthotic system according to claim 12, wherein the insertion element is adapted in such a way that the insertion element can be manually pushed and thus moved by a pressing force of at least 3 N so that its first part or second part of the ratchet system disengages from the positive locking with the respective first part or second part provided at the retainer.

15. The orthotic system according to claim 12, wherein a plurality of first parts and second parts are arranged in rows, respectively, so that the positive locking can be realized at different positions along the slide-in direction.

16. The orthotic system according to claim 13, wherein a plurality of first parts and second parts are arranged in rows, respectively, so that the positive locking can be realized at different positions along the slide-in direction.

17. The orthotic system according to claim 14, wherein a plurality of first parts and second parts are arranged in rows, respectively, so that the positive locking can be realized at different positions along the slide-in direction.
